(19) United States
(12) Patent Application Publication
(10) Pub. No.: US 2017/0097345 A1
(43) Pub. Date: Apr. 6, 2017

(54) ASSAY PLATE AND USES THEREOF

(71) Applicant: THE REGENTS OF THE UNIVERSITY OF MICHIGAN, Ann Arbor, MI (US)

(72) Inventors: Maung Kyaw Khaing Oo, Ann Arbor, MI (US); Xudong Fan, Ann Arbor, MI (US)

(21) Appl. No.: 15/280,093

(22) Filed: Sep. 29, 2016

Related U.S. Application Data
(60) Provisional application No. 62/235,795, filed on Oct. 1, 2015.

(51) Int. Cl. G01N 33/543; B01L 3/00

(52) U.S. Cl. CPC G01N 33/54386; B01L 3/502715; B01L 3/50273; B01L 2300/0829; B01L 2300/168; B01L 2300/04; B01L 2300/0627; B01L 2300/0887

(57) ABSTRACT

Provided herein are assay plates and uses thereof. In particular, provided herein are assay plates comprising optofluidic channels and microposts for use in performing biological and chemical assays and detecting assay results. The described plates provide reduced assay time and sample volume, and increased sensitivity and specificity in biological and chemical assays.

The present Application claims priority to U.S. Provisional Patent Application Ser. No. 62/235,795 filed Oct. 1, 2015, the disclosure of which is herein incorporated by reference in its entirety

### FIELD

Provided herein are assay plates and uses thereof. In particular provided herein are assay plates for performing biological and chemical assays and detecting assay results.

### BACKGROUND

A powerful and widely used current diagnostic technique, Enzyme-linked immunosorbent assay (ELISA), is demanded to improve sensitivity and reduce assay times. The main detection principles of current ELISA are based on Ultraviolet-visible absorption, chemiluminescence, and fluorescence detections. Drawbacks of traditional ELISA are: (1) long testing time (3-6 hours+overnight coating), which makes the ELISA almost useless when dealing with emergency care (such as heart attack, septic shock, traumatic brain injury, etc.) where the results should be obtained within 15-30 minutes; (2) large sample and reagent consumption (50-100 μL per sensor well), which adds significant costs to customers (˜$200 per test); and (3) inadequate detection limit, typically on the order of 10-100 pg/mL, which makes it impossible to measure many clinically significant biomarkers at low concentrations. All those drawbacks hinder the employment of ELISA in various applications that needs rapid, low cost, high sensitivity testing of trace quantity of analytes.

### SUMMARY

Provided herein are assay plates and uses thereof. In particular provided herein are assay plates for performing biological and chemical assays and detecting assay results.

For example, in some embodiments, the present disclosure provides an optofluidic array plate, comprising: a plurality of wells, wherein each well comprising a liquid inlet; an optically clear detection channel comprising a plurality of microposts; and a liquid outlet. In some embodiments, the inlet is offset from said detection channel. In some embodiments, the detection channel comprises a plurality of curves (e.g., symmetrical, U-shaped curves). In some embodiments, the inlet is a funnel. In some embodiments, the outlet is a nozzle. In some embodiments, the bottom surface of the array plate comprises a reflective layer (e.g., a metallic layer). In some embodiments, the array plate comprises or consists of 96 wells, although other sizes are specifically contemplated (e.g., 2, 4, 6, 24, 96, 384 or 1536 wells).

Additional embodiments provide a system, comprising: a) an array plate as described herein; and b) a bottom plate or film (e.g., adhesive film with die cut holes) configured to attach to the bottom of the array plate, wherein the bottom plate comprises a plurality of open-bottom fluidic outlets that correspond to each of the wells. In some embodiments, the array plate and the bottom plate are hermetically sealed. In some embodiments, the system further comprises a liquid transport pump. In some embodiments, the system further comprises a plurality of assay reagents (e.g., buffers, nucleic acid primers, nucleic acid probes, antibodies, or detection reagents). In some embodiments, the system further comprises a detection component (e.g., a plate reader or a spectrophotometer).

Further embodiments provide method of performing an assay, comprising: a) contacting a sample suspected of comprising an analyte with the system as described herein; and performing a detection assay with said system. In some embodiments, the analyte is a protein or a nucleic acid. In some embodiments, the assay is an immunoassay (e.g., ELISA), a nucleic acid amplification assay, or a nucleic acid hybridization assay. In some embodiments, the method detects the presence of said analyte in said sample.

Additional embodiments are described herein.

### DESCRIPTION OF THE FIGURES

The accompanying figures facilitate an understanding of the various, non-limiting embodiments of this technology.

FIG. 1A shows a top view of a micro-post array embedded optofluidic multi-well plate; FIG. 1B shows a bottom view thereof; FIG. 1C shows a front elevational view thereof; FIG. 1D shows a rear elevational view thereof; FIG. 1E shows a left side elevational view thereof; and FIG. 1F shows a right side elevational view thereof.

FIG. 2A shows an exploded perspective view of a micro-post array embedded optofluidic multi-well plate; FIG. 2B shows a perspective view of thereof; and FIG. 2C shows a perspective view with hidden line of thereof.

FIG. 3A shows an isometric view of Part A; and FIG. 3B shows a bottom view thereof.

FIG. 4A shows an isometric view of Part B; and FIG. 4B shows a bottom view thereof.

FIG. 5A shows a schematic detail of a micro-post array embedded optofluidic module; and FIG. 5B is a detail of micro-post array embedded optofluidic channel.

FIG. 6A illustrates rendered 3D image at top perspective view of a fully-assembled micro-post array embedded optofluidic multi-well plate; and FIG. 6B illustrates rendered 3D image at bottom perspective view thereof.

FIG. 7 shows photographs of manufactured (A) Part A, (B) Part B, and (C) die cut adhesive film.

FIG. 8 shows microscope images of 3×3 well (0.008 in×0.008 in channel) (A) an optofluidic module, and (B) micro-post (arrows) array layout.

FIG. 9 shows photographs of manufactured Part A (0.018 in×0.022 in channel), (A) Front, and (B) Back views of clear and transparent optofluidic well plate.

FIG. 10 shows photographs of manufactured Part A (0.018 in×0.022 in channel), (A) Front, and (B) Back views of black and opaque optofluidic well plate.

FIG. 11 shows microscope images of an optofluidic module of 3×3 well (0.018 in×0.022 in channel) and micro-post (arrows) array layout.

FIG. 12 shows a photograph of 3D printed well plate adapter (black).

FIG. 13 shows a comparison of run-to-run variations of three different well plates; (1) a conventional 96-well plate, (2) an OPTIMISER™ well plate, and (3) an optofluidic well plate of embodiments of the present disclosure.

FIG. 14 shows a comparison of well-to-well variations of three different well plates; (1) a conventional 96-well plate, (2) an OPTIMISER™ well plate, and (3) an optofluidic well plate of embodiments of the present disclosure.

FIG. 15 shows a comparison of fluorescence intensities of 0.5 μM Rhodamine 6G with different channels sizes.

FIG. 16 shows a cross talk analysis of (A) fluorescence using a clear and transparent well plate, (B) chemiluminescence using a clear and transparent well plate, and (C) chemiluminescence using a black and opaque well plate.

FIG. 17 shows a standard curve of IL-6 in buffer using fluorescence detection method and statistical analysis of clear and transparent optofluidic well plate (0.008 in×0.008 in channel) of embodiments of the present disclosure; (A) three data point from each concentration with four parameter logic (4-PL) curve fit, (B) average values with standard deviations of three wells, (C) coefficient of variation, and (D) p value of each IL-6 concentration.

FIG. 18 shows a standard curve of IL-6 in buffer using fluorescence detection method and statistical analysis of clear and transparent optofluidic well plate (0.018 in×0.022 in channel) of embodiments of the present disclosure; (A) three data point from each concentration with four parameter logic (4-PL) curve fit, (B) average values with standard deviations of three wells, (C) coefficient of variation, and (D) p value of each IL-6 concentration.

FIG. 19 shows a standard curve of IL-6 in serum using fluorescence detection method and statistical analysis of clear and transparent optofluidic well plate (0.018 in×0.022 in channel) of embodiments of the present disclosure; (A) three data point from each concentration with four parameter logic (4-PL) curve fit, (B) average values with standard deviations of three wells, (C) coefficient of variation, and (D) p value of each IL-6 concentration.

FIG. 20 shows a standard curve of IL-6 in buffer using chemiluminescence detection method and statistical analysis of black and opaque optofluidic well plate (0.018 in×0.022 in channel) of embodiments of the present disclosure; (A) three data point from each concentration with four parameter logic (4-PL) curve fit, (B) average values with standard deviations of three wells, (C) coefficient of variation, and (D) p value of each IL-6 concentration.

FIG. 21 shows a standard curve of IL-6 in serum using chemiluminescence detection method and statistical analysis of black and opaque optofluidic well plate (0.018 in×0.022 in channel) of embodiments of the present disclosure; (A) three data point from each concentration with four parameter logic (4-PL) curve fit, (B) average values with standard deviations of three wells, (C) coefficient of variation, and (D) p value of each IL-6 concentration.

FIG. 22 shows a comparison of conventional 96-well plate and optofluidic well plate (0.018 in×0.022 in channel); (A) standard curves of IL-6 in buffer with conventional 96-well, plate and clear and transparent optofluidic well plate using fluorescence detection method, (B) standard curves of IL-6 in serum with conventional 96-well plate, and clear and transparent optofluidic well plate using fluorescence detection method, (C) standard curves of IL-6 in buffer with conventional 96-well plate (CORNING™ 96-Well Clear Bottom Black Polystyrene Microplate), and black and opaque optofluidic well plate using chemiluminescence detection method, and (D) standard curves of IL-6 in serum with conventional 96-well plate (CORNING™ 96-Well Clear Bottom Black Polystyrene Microplate), and black and opaque optofluidic well plate using chemiluminescence detection method.

### DEFINITIONS

To facilitate an understanding of the present disclosure, a number of terms and phrases are defined below, or terms may be defined elsewhere in the disclosure:

The term “sample” is used in its broadest sense. On the one hand it is meant to include a specimen or culture. On the other hand, it is meant to include both biological and environmental samples.

Biological samples may be animal, including human, fluid, solid (e.g., stool) or tissue, as well as liquid and solid food and feed products and ingredients such as dairy items, vegetables, meat and meat by-products, and waste. Biological samples may be obtained from the various families of domestic animals, as well as feral or wild animals, including, but not limited to, such animals as ungulates, bear, fish, lagamorphs, rodents, etc. or combinations thereof.

Environmental samples include environmental material such as surface matter, soil, water and industrial samples, as well as samples obtained from food and dairy processing instruments, apparatus, equipment, utensils, disposable and non-disposable items or combinations thereof. These examples are not to be construed as limiting the sample types applicable to the present disclosure.

As used herein, the term “in vitro” refers to an artificial environment and to processes or reactions that occur within an artificial environment. In vitro environments can consist of, but are not limited to, test tubes and/or cell culture. The term “in vivo” refers to the natural environment (e.g., an animal or a cell) and to processes or reactions that occur within a natural environment.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds. A test compound may be determined to be therapeutic by screening using the screening methods, devices, and/or systems of the present disclosure. In certain embodiments of the present disclosure, test compounds may include antisense, siRNA and/or shRNA compounds.

The term “spheroid” refers to clusters or aggregates of cells and/or cell colonies.

Spheroids may be formed from various cell types, for example, primary cells, cell lines, tumor cells, stem cells, etc. Spheroids may have sphere-like or irregular shapes. Spheroids may contain heterogeneous populations of cells, cell types, cells of different states, such as proliferating cells, quiescent cells, and necrotic cells.

### DETAILED DESCRIPTION

The following description is provided in relation to several embodiments which may share common characteristics and features. It is to be understood that one or more features of one embodiment may be combinable with one or more features of the other embodiments. In addition, a single feature or combination of features in any of the embodiments may constitute additional embodiments.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The subject headings used in the detailed description are included only for the ease of reference of the reader and should not be used to limit the subject matter found throughout the disclosure or the claims. The subject headings should not be used in construing the scope of the claims or the claim limitations.

Provided herein are assay plates and uses thereof. In particular provided herein are assay plates for performing biological and chemical assays and detecting assay results. In some embodiments, the plates comprise multi (e.g., 96) optofluidic modules that conform to the dimensions of a standard 96-well or other size plates. Thus, the plate can be used to measure fluorescence, luminescence, Raman scattering, surface enhanced Raman scattering, and absorption with any available standard plate readers in the market. The present disclosure is not limited to a 96-optofluidic well plate. In some embodiments, the design is customized based on customer requirements such as desired number of optofluidic modules and their positions, and the size of individual optofluidic module. Furthermore, in some embodiments, a reflective layer is coated on an outside surface of the plate to improve optical detection efficiency.

The plates described herein provide significant advantages over existing technologies. For example, placing the inlet away from the detection region avoids potential interference with optical measurements and any residual liquid left in the inlet. This significantly reduces measurement variability and improves signal intensity (See experimental section). In addition, symmetrical channels allow a large tolerance in optical detection position without signal variation. The inclusion of posts within the detection channels increases surface to volume ratio and/or mass transport rate to improve analyte capture efficiency and the total number of captured analytes. This reduces overall assay time and increases signal intensity. In addition, each post may work as an optical waveguide to direct the light to the light detector, which further increases signal intensity. The addition of an outlet nozzle and optional pump improves flow inside the fluid channel, which reduces assay time and measurement variation. The inclusion of an optional reflective layer at the bottom of the assay plate reflects light back to the detector and increases signal.

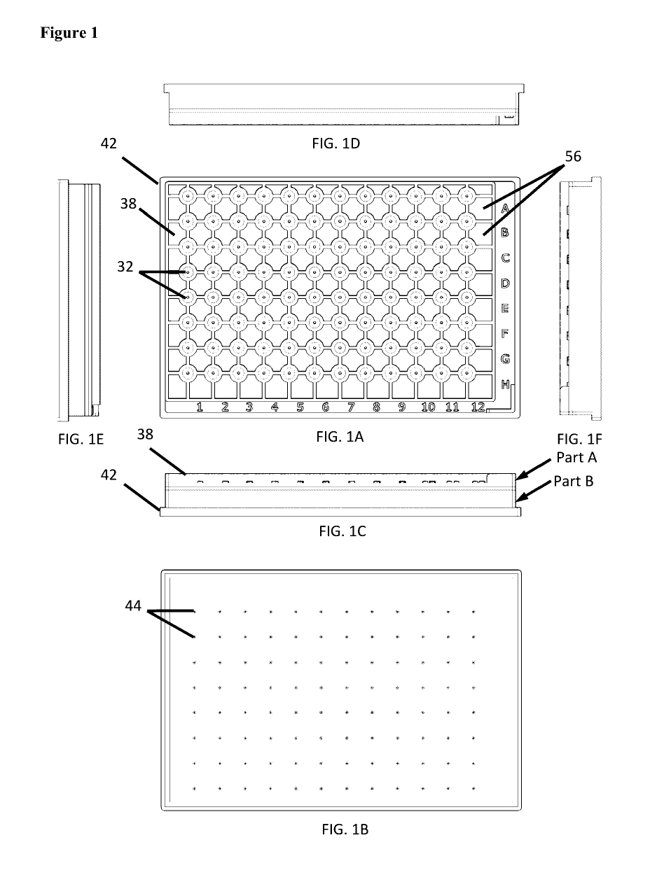

Exemplary plates are shown in FIGS. 1-12. The plates illustrated in the Figures utilize a 96 well configuration, although other configurations are contemplated. The plate has two parts, a top, Part A and a bottom, Part B. They are attached together and hermetically sealed with adhesive film or glue or using ultrasonic plastic welding technique or other suitable method. Alternatively, the Part B can be replaced with adhesive film with die cut holes. In the top Part A, 96 optofluidic modules are arranged and positioned in 8×12 format array for reading with market available standard plate readers. Each module has a fluidic inlet and a loop of optofluidic channel. 96 open-bottom fluidic outlets are located in the bottom of Part B or die cut adhesive film. Microstructured posts are systematically arranged in the loop of optofluidic channel that connects to the inlet and the outlet. In some embodiments, the plate's footprint, height, bottom and outside flange are adapted from ANSI (the American National Standards Institute)/SLAS (Society for Laboratory Automation and Screening) 96-well plate standard. During the measurement, reagents and samples are added from the inlet, then flow through the loop channel, and eventually drain from the outlet by a wicking method or pressure differential. Optical signal can be detected at the center of the loop of channel without adjusting standard micro plate reader.

The dimension of each optofluidic module can be scaled either smaller or larger than that in this disclosure. In some embodiments, an optional reflective layer is coated on the surface of the plate on the opposite side of detection (e.g., to improve optical detection efficiency).

The advantages of this design are: (1) It is completely compatible with standard well plate readers; (2) The microstructured post increases the surface-to-volume ratio and/or mass transport rate, which improves analyte capture efficiency and the total number captured analytes, and boost sensitivity; (3) Flow-through design simplifies the sample (solution) addition and withdrawal for reduced assay time; (4) Part of emitted light is guided and accumulated along the longitudinal direction of the microstructured post that enhances signal collection and hence sensitivity; and (5) (Optional) The collection efficiency can be further increased using reflective coating or reflective mirror at an outside surface of the plate.

### I. Plates

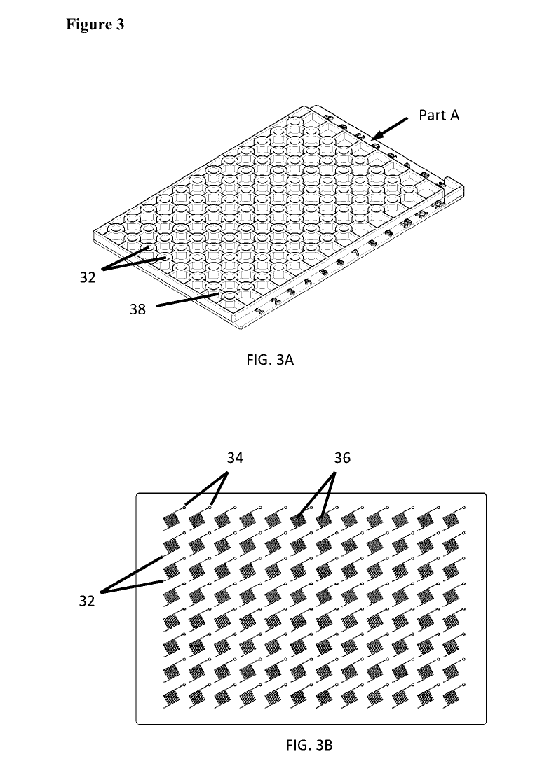

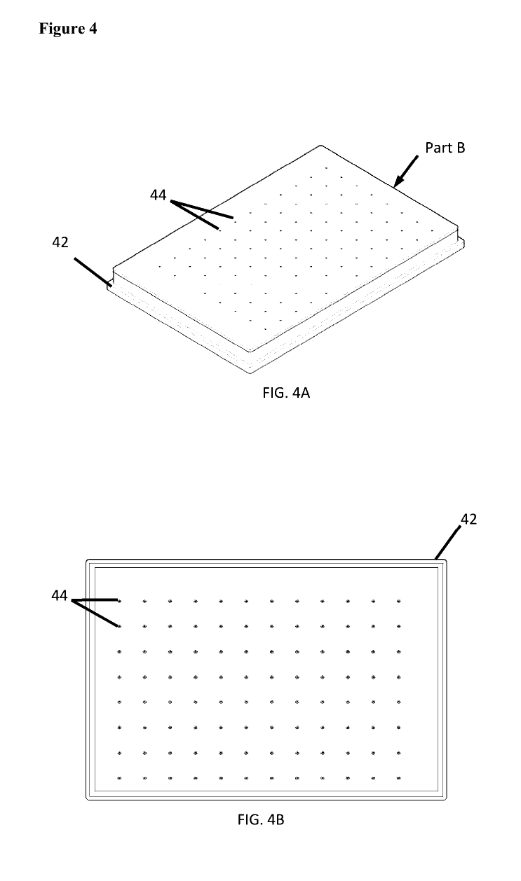

FIG. 1A-F exhibit a micro-post array embedded optofluidic multi-well plate. In some embodiments, plates are shown as standard round wells, although other shapes and sizes of wells are specifically contemplated (e.g., larger or smaller wells than ANSI standards or square or abstract shaped wells). The Figure shows (1) funnel-shape-well attached micro-post array embedded optofluidic modules, Part A, and (2) drain holes or adhesive film with die cut holes, Part B. The optofluidic outlets 34 (in FIG. 3), are well aligned with drain hole 44 (in FIG. 4). The plate's footprint, height, bottom and outside flange, and well position dimensions are adapted from ANSI (the American National Standards Institute)/SLAS (Society for Laboratory Automation and Screening) 1-2004, 2-2004, 3-2004, and 4-2004 respectively. Thus, the plate is compatible with any standard plate reader. The outside dimensions of the base footprint length and width of the plate are 5.0299 and 3.3654 inches respectively. The height of the plate is 0.565 inch. The plate has a surrounding skirt 42, a top surface 38 and an array of funnel-shape-wells 32 attached to the micro-post array embedded optofluidic channels 36 (in FIG. 3). The detection areas 56 are arranged in 12 rows with 8 columns of standard micro plate reader excitation and collection positions. Centers of adjacent detection areas are spaced apart 0.3542 inch. The optofluidic 96-well can be adopted for any customized number of wells format in a similar manner described in this disclosure. In some embodiments, Part A and Part B are formed by injection molding and made of plastic (e.g., polystyrene, PMMA (Poly(methyl methacrylate)). In some embodiments, they are bonded and hermetically sealed with adhesive film or glue or using ultrasonic plastic welding technique or other suitable method.

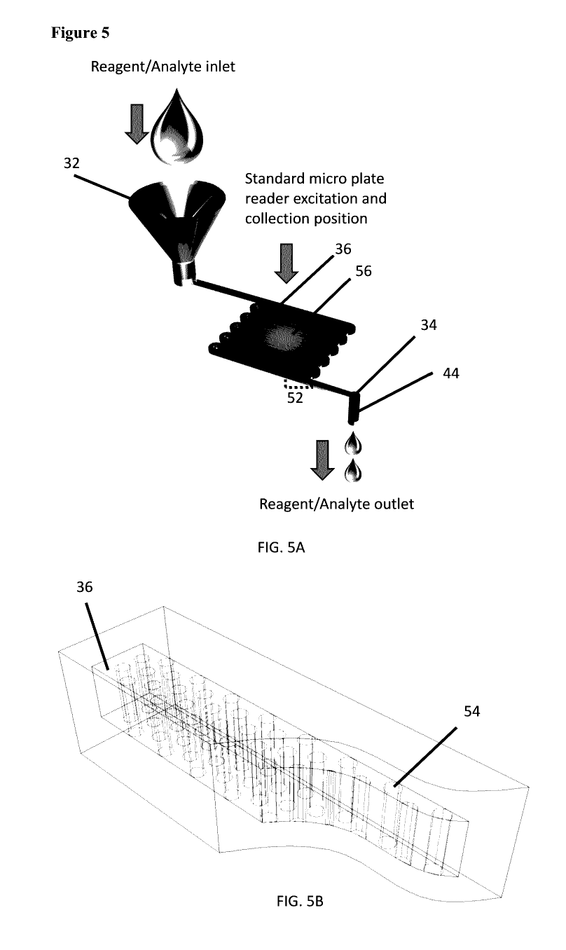

The inlets 32 are shown in some embodiments as funnel shaped, although other shapes are specifically contemplated. The inlet may be funnel shaped as shown in FIG. 5A, cylindrical, triangular, inverted funnel, or other configurations. Any configuration that allows reagents, samples, etc. to enter the wells may be utilized.

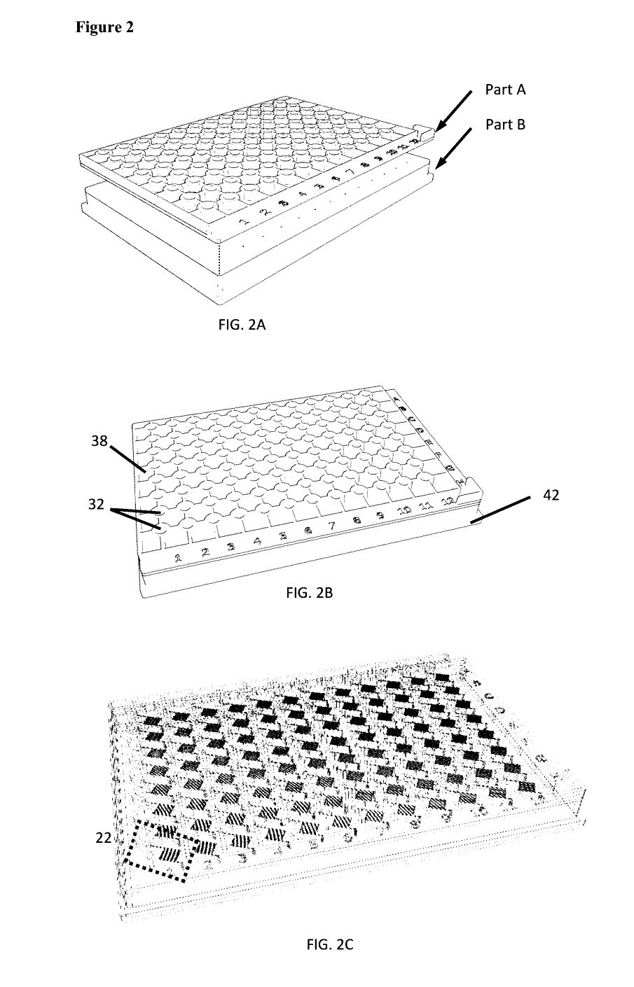

FIG. 2A, FIG. 2B, and FIG. 2C show an exploded perspective view, a perspective view, and a perspective view with hidden lines of the present disclosure. The Part A and Part B are stacked and aligned as shown in FIG. 2A, and then bonded together as shown in FIG. 2B. The array of 96 individual optofluidic modules 22 can be seen in FIG. 2C.

FIG. 3A is an isometric view of Part A, and FIG. 3B is a bottom view of it. The array of optofluidic modules are arranged in 12 rows with 8 columns. Centers of adjacent modules are spaced apart 0.3542 inch. Each module has funnel-shape-well 32, which has a depth of 0.118 inch, diameters of 0.118 inch at the entrance, and 0.039 inch at the exit that located next to the entrance of micro channel 36. The microchannels are not limited to particular sizes and shapes.

In some embodiments, the channels are circular, oval, square or another shape and are approximately 0.001-0.1 inch in diameter. In some embodiments, microchannels are arranged in a series (e.g., at least 1, 2, 3, 4, 5, 10, 50, or more) U or S shaped curves, although other configurations are specifically contemplated, depending on the application. The other end of the micro-post array embedded loop optofluidic channel 36 has an opening 34 with diameter of 0.02 inch. Other sizes are specifically contemplated based on space and assay configuration. The funnel-shape-well 32 on the top layer of the Part A delivers samples/reagents to the corresponding optofluidic channel 36 with easy, convenient, and efficient way.

FIG. 4A is an isometric view of Part B and FIG. 4B is a bottom view of it. The Part B of the skit 42 fits any standard plate readers. Cylindrical tubes 44 with heights of 0.078 inch, inner diameters of 0.02 inch, and outer diameters of 0.059 inch, are arranged in 12 rows with 8 columns. They are aligned with fluid exit openings of Part A.

FIG. 5A is a schematic detail of a micro-post array embedded optofluidic module 22. Samples and reagents can flow from the funnel-shape-well 32 through the micro-post array embedded loop optofluidic channel 36. Those fluids can be withdrawn from the opening 34 and the cylindrical tube 44 or adhesive film with die cut holes using a wicking method or pressure differential. The optical signal can be acquired at the center of the optofluidic loop 56 located at standard micro plate reader optical excitation and collection position. FIG. 5B is a detail of micro-post array embedded optofluidic channel at the section 52 of the loop optofluidic channel 36. The depth and width of optofluidic channel 36 are 0.008 inch and 0.008 inch. Between adjacent two channels has a 0.008 inch thick wall. In the channel 36, micro posts 54 which have 0.002 inch in diameter and 0.008 inch in height (the same as the depth of the channel 34) are located 0.003 inch apart. The desired range of depth and width of the channel, height, and diameter of the post, and allocation of the post (See e.g., Examples) are adjusted based on manufacturing feasibility and use.

A plurality of posts 54 are shown. The present disclosure is not limited to the number, size, or shape of the posts. In some embodiments, each channel 36 has from 10-1000 (e.g., 20-750, 50-500, or 50-250 posts 54). In some embodiments, the posts 54 are cylindrical, prism, rectangular, trapezoidal, or other shape). In some embodiments, the posts 54 are approximately 0.002 to 0.004 inch in diameter and 0.0002 to 0.008 inch in height, although other sizes are specifically contemplated. In some embodiments, the posts serve to increase sensitivity of the assay by providing additional binding locations for assay reagents (e.g., antibodies or nucleic acids).

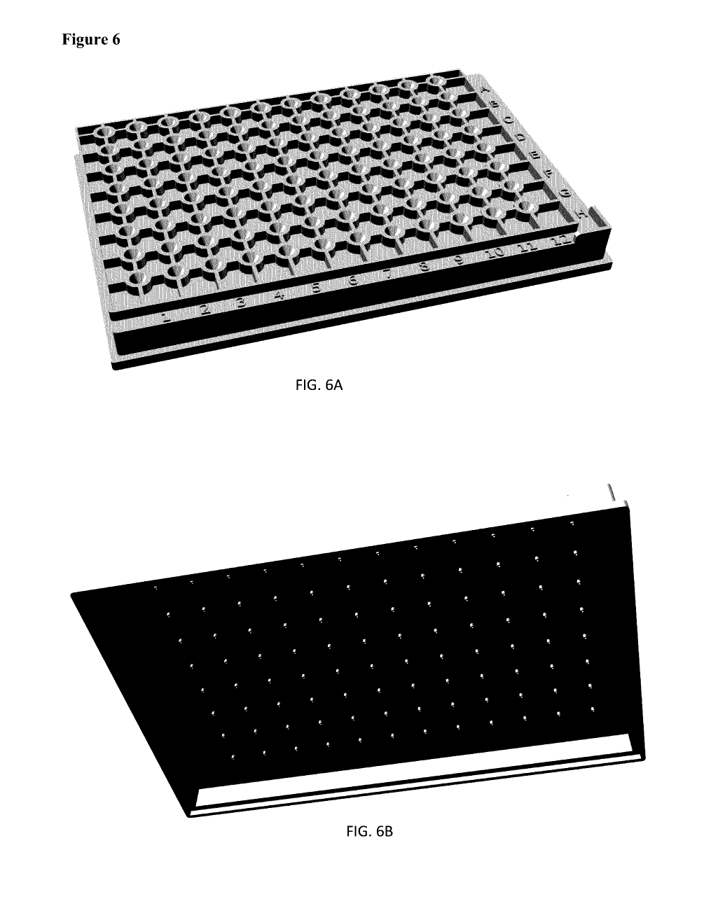

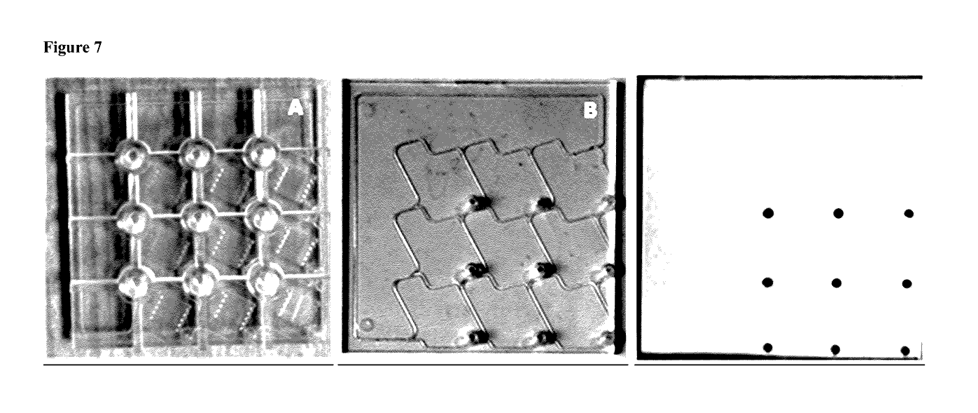

FIGS. 6A and 6B illustrate the visual appearance of a fully-assembled micro-post array embedded optofluidic multi-well plate at a top perspective view and a bottom perspective view respectively.

In some embodiments, the present disclosure provides systems and/or kits comprising devices (e.g., comprising assay plates), alone or in combination with reagents for performing assays (e.g., nucleic acid primers and probes, antibodies, detection reagents, buffers, test compounds, controls, etc.). In some embodiments, systems and kits comprise robotics for use in high throughput analysis (e.g., sample handling and analysis (e.g., plate readers) equipment). In some embodiments, systems further comprise detection components (e.g., plate readers and spectrophotometers)

### II. Uses

The assay plate devices of certain embodiments of the present disclosure find use in a variety of applications (e.g., diagnostic, screening, and research applications). In some embodiments, assays are performed to determine the presence of an analyte in a sample (e.g., biological sample). A variety of nucleic acid and amino acid detections assays can be performed in the assay plates. Exemplary assays are described below.

In some embodiments, reagents (e.g., capture nucleic acids or antibodies) are added through the sample inlet. Reagents then adhere to the micro posts and channel walls. Next, sample (e.g., test samples suspected of containing an analyte) are added via the inlet. Excess reagents are removed via the outlet. Once the assay is completed, a plate reader or spectrometer is used to visualize assay in the channel results through the well. In some embodiments, computer systems and/or computer software is used to determine and report assay results (e.g., presence of analytes in the test sample) to the user via a user interface (e.g., computer screen, tablet, smart phone, etc.). In some embodiments, the results are used in research, diagnosis, or determining a treatment course of action.

Illustrative non-limiting examples of immunoassays include, but are not limited to: immunoprecipitation; Western blot; ELISA; immunohistochemistry; immunocytochemistry; immunochromatography; flow cytometry; and, immuno-PCR. Polyclonal or monoclonal antibodies detectably labeled using various techniques known to those of ordinary skill in the art (e.g., colorimetric, fluorescent, chemiluminescent or radioactive labels) are suitable for use in the immunoassays.

Immunoprecipitation is the technique of precipitating an antigen out of solution using an antibody specific to that antigen. The process can be used to identify proteins or protein complexes present in cell extracts by targeting a specific protein or a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride, or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and optionally sorting microscopic particles or cells suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

Exemplary nucleic acid detection methods that can be performed in the assay plates described herein include, but are not limited to, sequencing assays, amplification assays, and hybridization assays.

Illustrative non-limiting examples of nucleic acid sequencing techniques include, but are not limited to, chain terminator (Sanger) sequencing and dye terminator sequencing, or high throughput sequencing methods. The present disclosure is not intended to be limited to any particular methods of sequencing. Those of ordinary skill in the art will recognize that because RNA is less stable in the cell and more prone to nuclease attack experimentally RNA is usually reverse transcribed to DNA before sequencing.

In some embodiments, the sequencing is the real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10×10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

A number of other DNA sequencing techniques can be used, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties) and the Adessi PCR colony technology (Adessi et al. (2000). Nucleic Acid Res. 28, E87; WO 00018957; herein incorporated by reference in its entirety).

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing in employed (see, e.g., Astier et al., J Am Chem Soc. 2006 Feb. 8; 128(5):1705-10, herein incorporated by reference). The theory behind nanopore sequencing has to do with what occurs when the nanopore is immersed in a conducting fluid and a potential (voltage) is applied across it: under these conditions a slight electric current due to conduction of ions through the nanopore can be observed, and the amount of current is exceedingly sensitive to the size of the nanopore. If DNA molecules pass (or part of the DNA molecule passes) through the nanopore, this can create a change in the magnitude of the current through the nanopore, thereby allowing the sequences of the DNA molecule to be determined.

In certain embodiments, HeliScope by Helicos BioSciences is employed (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In certain embodiments, the Ion Torrent technology (Life Technologies) is employed to sequence purified target nucleic acid sequences. The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

Another exemplary nucleic acid sequencing approach that may be adapted for use with the present disclosure was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Patent Publication No. 20090035777.

Other emerging single molecule sequencing methods include real-time sequencing by synthesis using a VisiGen platform (Voelkerding et al., Clinical Chem., 55: 641-658, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with radio-, fluorescent- or antigen-labeled bases is localized and quantitated in the tissue using autoradiography, fluorescence microscopy or immunohistochemistry. ISH can also use two or more probes, labeled with radioactivity or the other non-radioactive labels, to simultaneously detect two or more transcripts.

Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782, each of which is herein incorporated by reference in its entirety.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in method of embodiments of the present disclosure. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products methods of embodiments of the present disclosure. See, e.g., U.S. Pat. No. 5,814,447 (herein incorporated by reference in its entirety).

### EXPERIMENTAL

The following examples are provided in order to demonstrate and further illustrate certain preferred embodiments and aspects of the present disclosure and are not to be construed as limiting the scope thereof.

### Example 1

### Optofluidic Well Plate Fabrication (0.008 in Width×0.008 in Depth Channel with 0.002 in Diameter Micro Post Array)

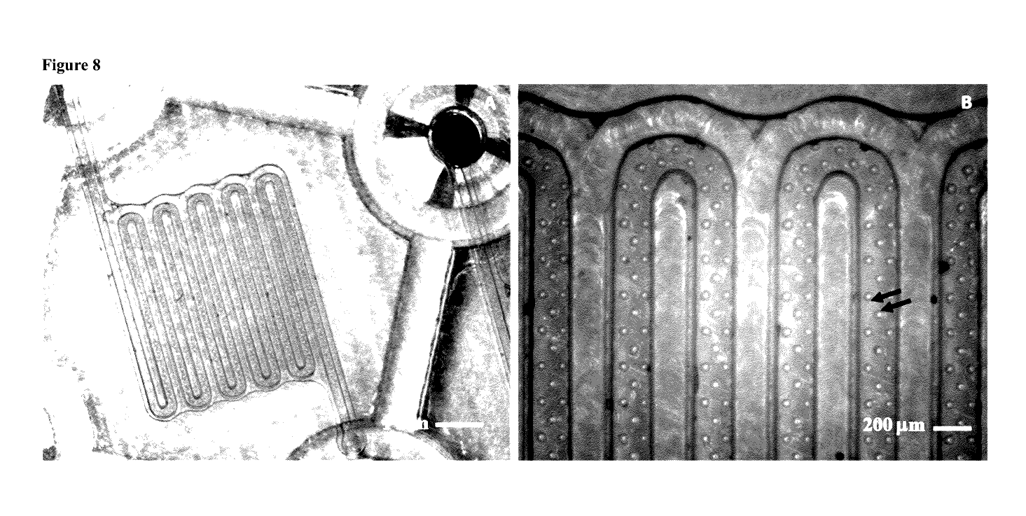

A 3×3 (3 rows with 3 columns) well lay-out of Part A (FIG. 7A) and Part B (FIG. 7B) of micro-post array embedded optofluidic multi-well plate were manufactured using polystyrene materials with injection mold. Alternatively, one-sided adhesive film with 9 die cut holes (FIG. 7C) was used as Part B for further experiments. FIGS. 8A and B exhibit over all layout of an optofluidic module and micro-post array inside the channel of the Part A respectively.

### Optical Performance Study: Variations of Optical Detections

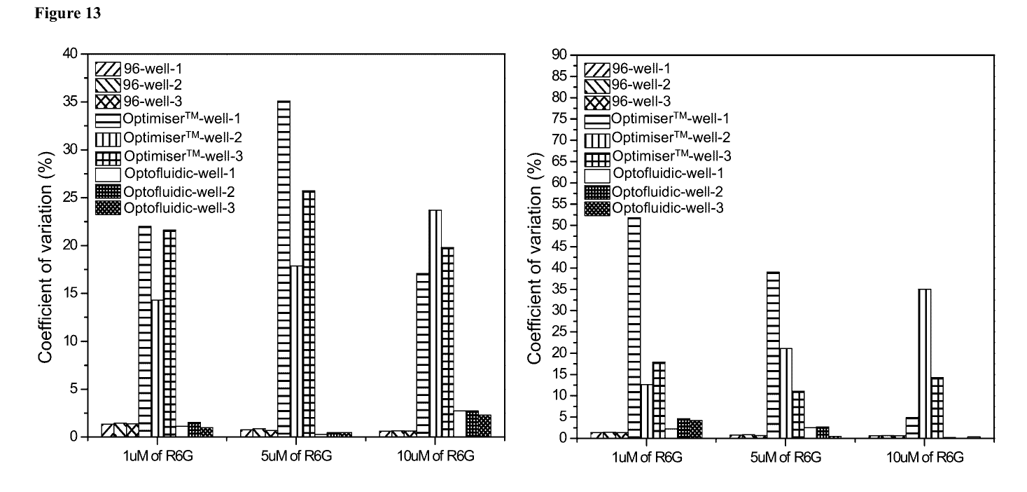

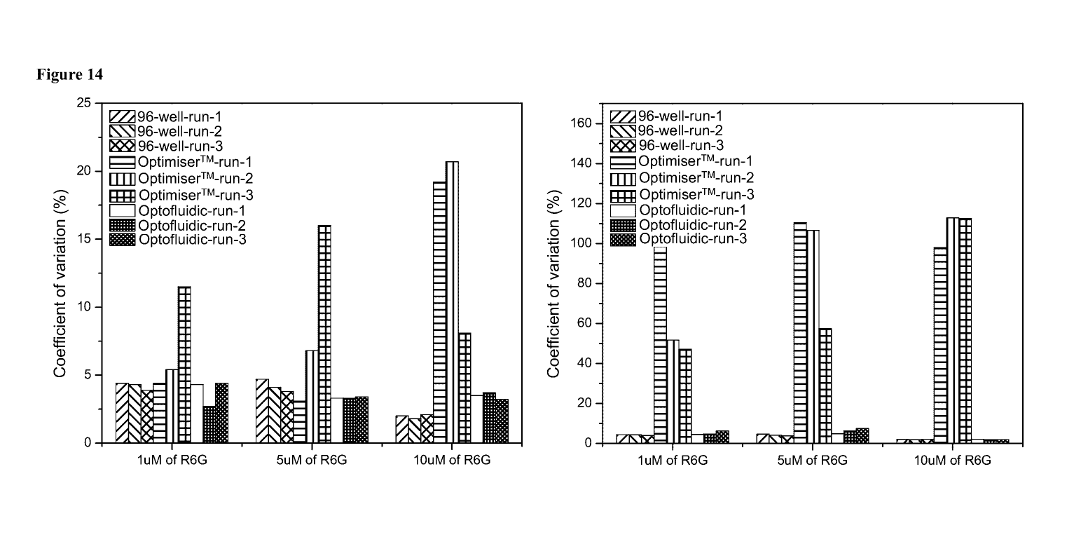

In order to evaluate optical signal detection variations, the optofluidic well plate of present disclosure performance against conventional 96-well and OPTIMISER™ well plates were compared (FIGS. 13 and 14.

### ELISA Study

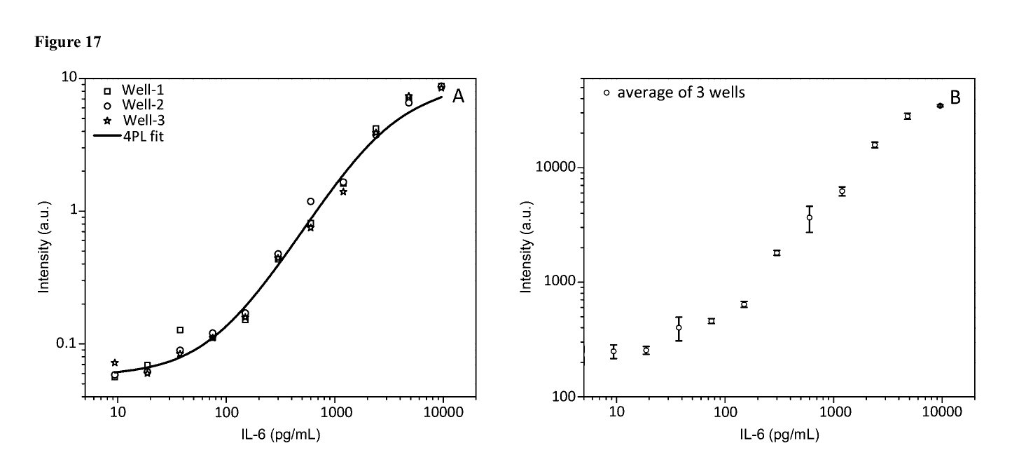

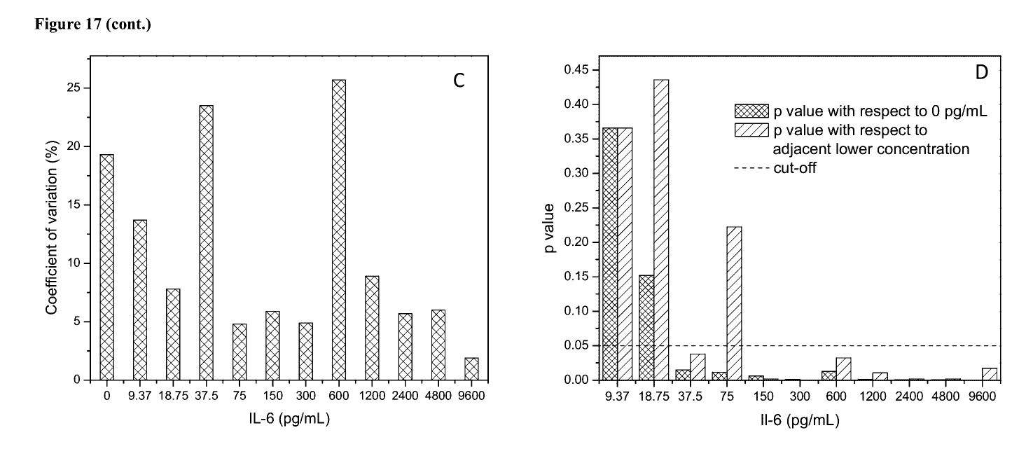

Various concentration of Human IL-6 dissolved in buffer solutions were tested with optofluidic well plates (FIG. 17). See more details in the ELISA protocol section.

### Example 2

### Optofluidic Well Plate Fabrication (0.018 in Width×0.022 in Depth Channel with 0.004 in Diameter Micro Post Array)

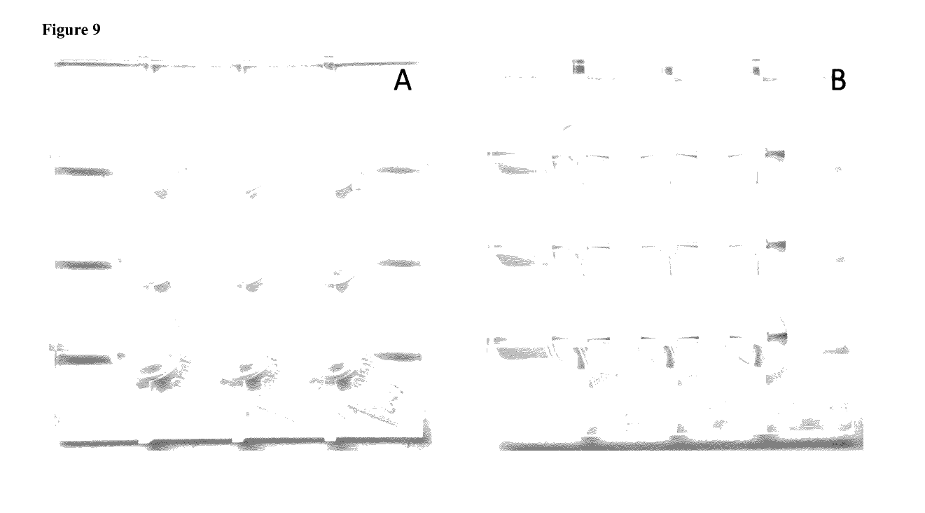

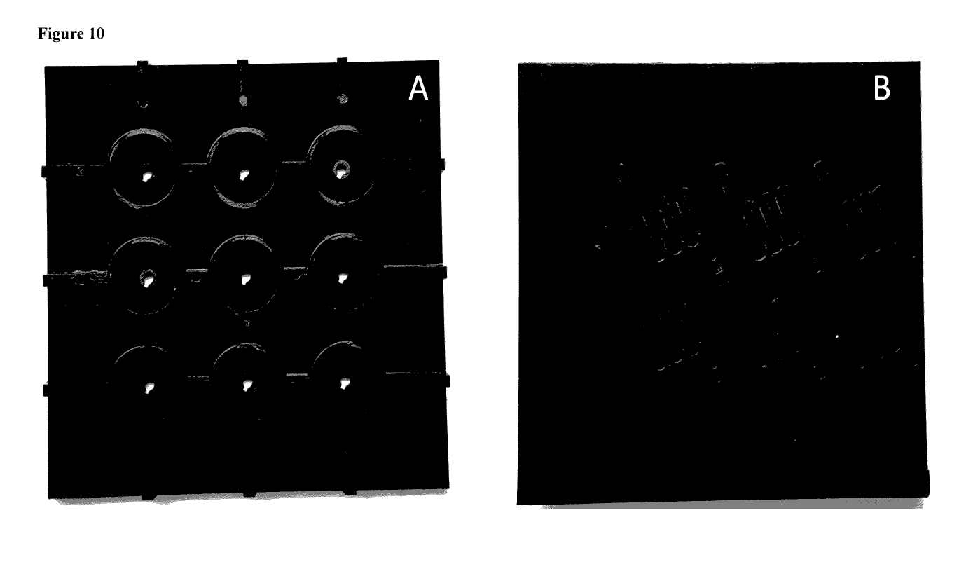

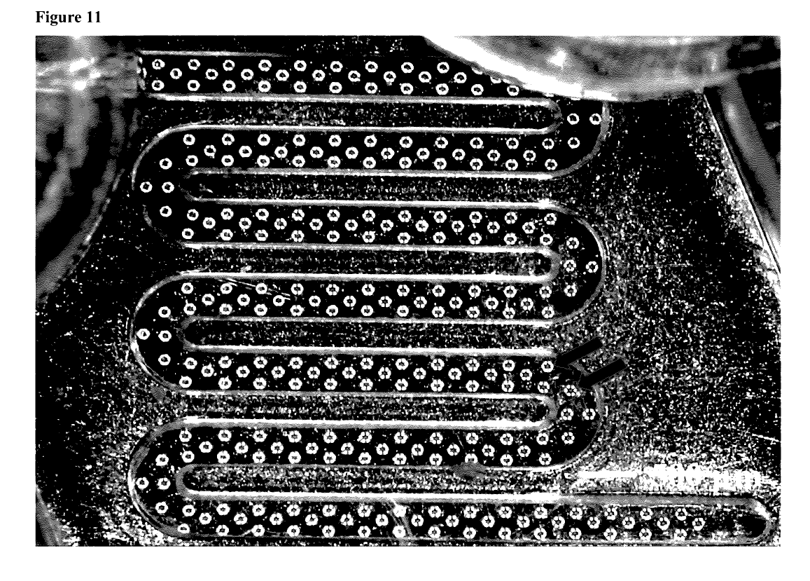

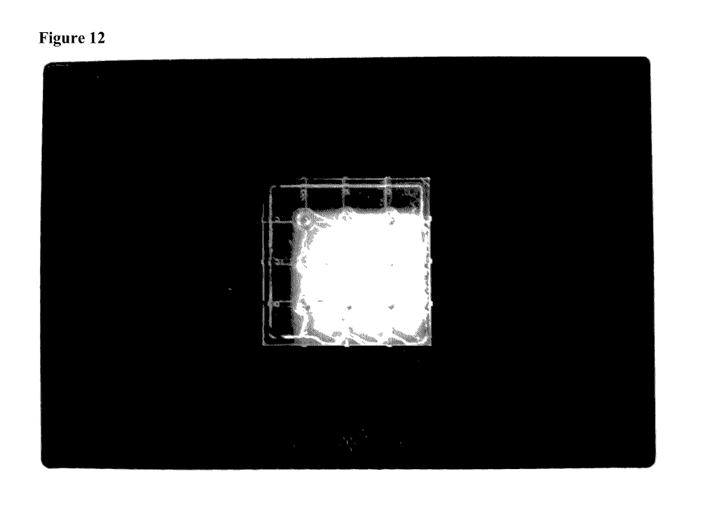

A 3×3 (3 rows with 3 columns) well lay-out of Part A of micro-post array embedded optofluidic multi-well plate was manufactured using polystyrene materials with injection mold (clear and transparent well plate as shown in front image of FIG. 9A and back image of FIG. 9B and black and opaque well plate as shown in front image of FIG. 10A and back image of FIG. 10B). One-sided adhesive film with 9 die cut holes (FIG. 7C) was used as Part B with these Part A of optofluidic well plates for further experiments. FIG. 11 exhibits micro-post array inside the channel of the Part A. In order to fit Standard Multilabel Plate Reader with 3×3 well plate, 3D printed well plate adapter as shown in FIG. 12 was used.

### Optical Performance Study: Dependent of Channel Size

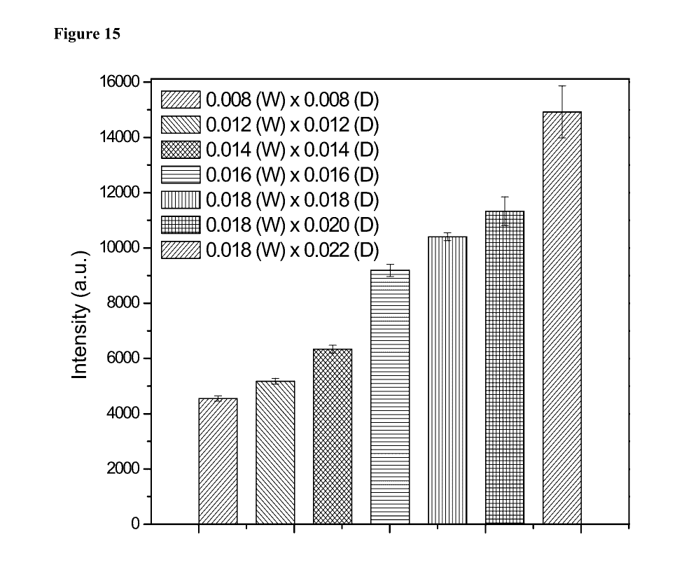

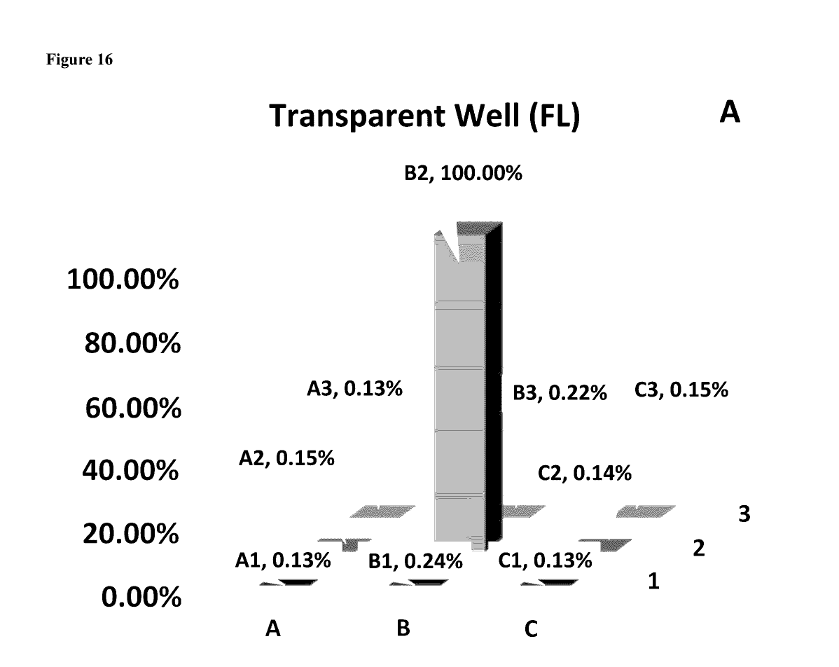

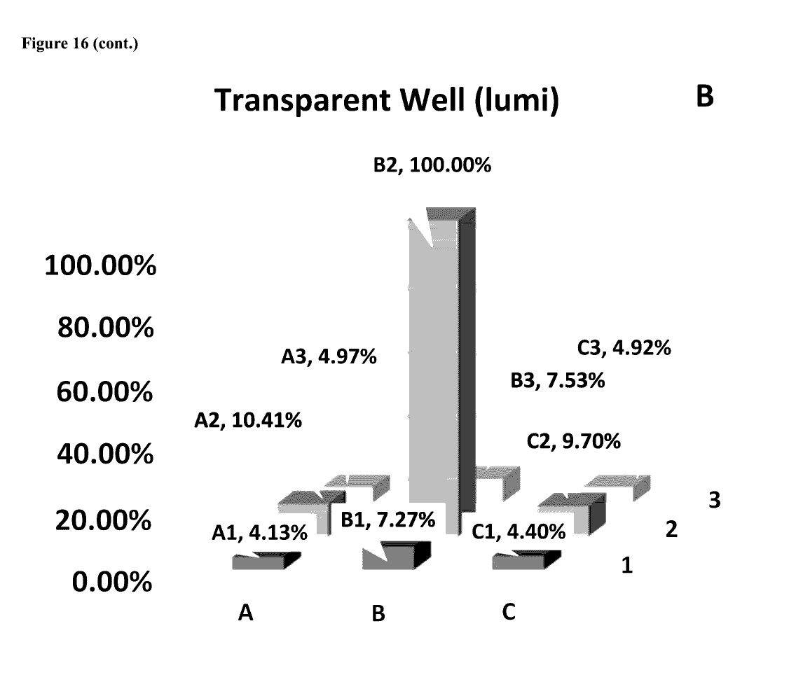

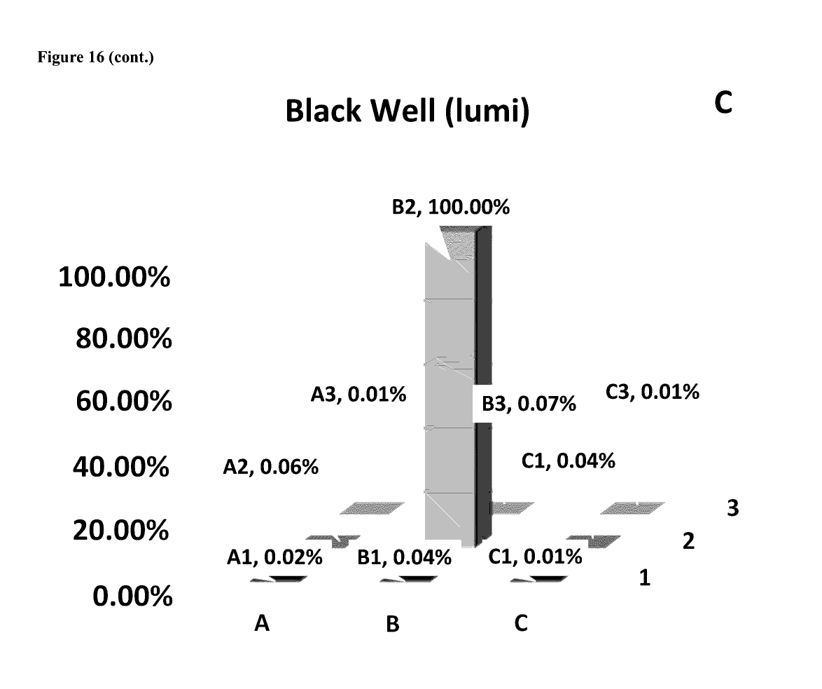

In order to evaluate optical performance of the optofluidic well plate of the present disclosure, seven different channel sizes (FIG. 15) were studied using Rhodamine 6G (R6G) dissolved in methanol as described in the materials and methods section. In addition, optical cross talks of clear and transparent, and black and opaque polystyrene optofluidic well plates were analyzed (FIG. 16).

### ELISA Study

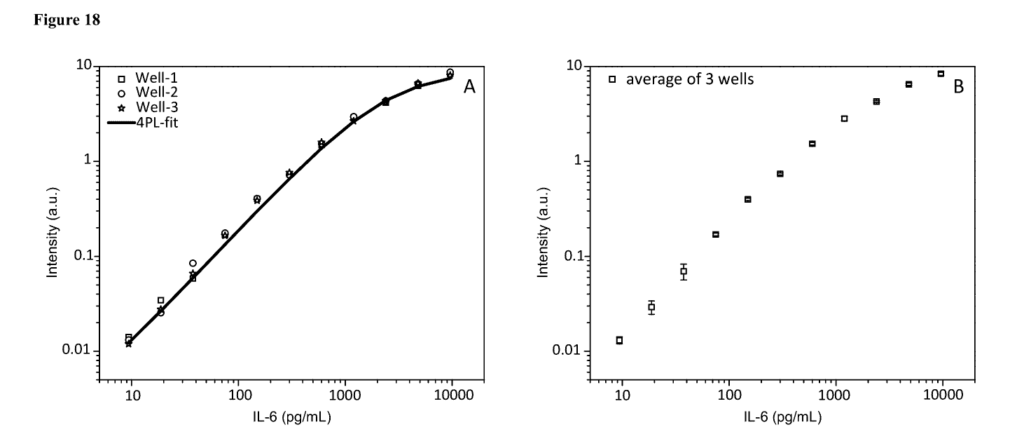

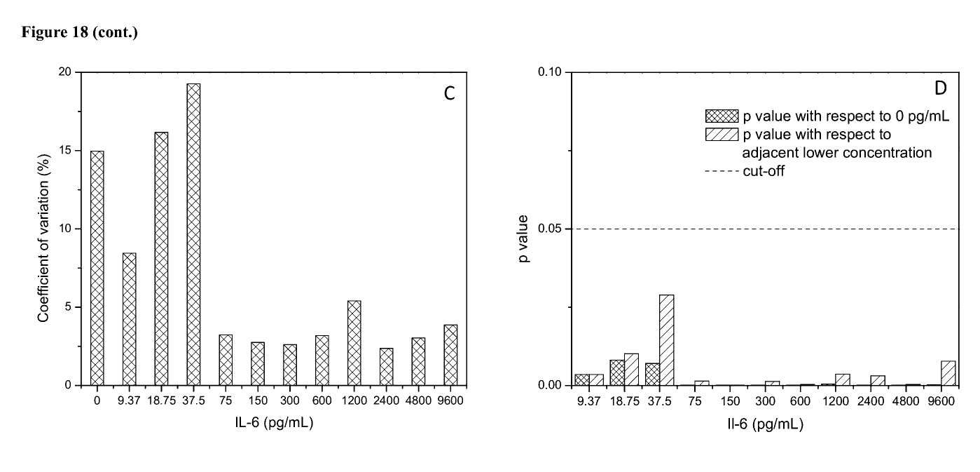

Various concentration of Human IL-6 dissolved in buffer solutions as well as serum were tested with optofluidic well plates by two detection methods: 1. Fluorescence detection with clear and transparent well plate; and 2. Chemiluminescence detection with black and opaque well plate. See more details in the ELISA protocol section. Conventional 96-well plates were tested with the same Human IL-6 concentrations that were used in the optofluidic well plate, using conventional ELISA protocol of R&D Systems for benchmarking (FIG. 18-22).

### Materials and Methods for Optical Performance Study

Rhodamine 6G (R6G) powder (Sigma-Aldrich #252433) was thoroughly dissolved in 99.8% methanol (Sigma-Aldrich #322415) to construct 1 mM concentration. Then, the solution was diluted sequentially to make desired concentrations. Those solutions were filled into desired amount into the wells or channels. 100 μL of R6G solution was used in conventional 96-well plate. Both 10 μL and 3 μL of R6G solution were used in OPTIMISER™ well plate, and optofluidic well plate.

Standard Multilabel Plate Reader (Perkin Elmer EnSpire® 2300) was used to acquire fluorescence intensity. Excitation wavelength of 480 nm with <8 nm bandwidth, emission wavelength of 550 nm with <8 nm bandwidth, and excitation flash intensity of 100 were used. Fluorescence intensity readings were taken from above the well plates. The height of measurement head which consists of excitation and emission channels, was adjusted to gain maximum sensitivity of each type of well plate. The heights of 3.8 mm, 11.1 mm, and 7.5 mm, were used in conventional 96-well, OPTIMISER™ well plate, and optofluidic well plate, respectively. Triplicate samples (3 wells) of each well plate were read three times (3 runs) and calculated coefficient of variations (CVs) of wells in each run and CVs of runs in each well.

For the channel size dependent of fluorescence intensity study, 0.5 μM of R6G solution was used with 7 different channel sizes; (1) 0.008 in wide×0.008 in depth, (2) 0.012 in wide×0.012 in depth, (3) 0.014 in wide×0.014 in depth, (4) 0.016 in wide×0.016 in depth, (5) 0.018 in wide×0.018 in depth, (6) 0.018 in wide×0.020 in depth, and (7) 0.018 in wide×0.022 in depth.

### Materials and Method for Surface Modification of Optofluidic Well Plate

The optofluidic well plates were made of polystyrene, which has hydrophobic surface nature, making it impossible to flow reagents through the micron sized channels. Therefore, the well plates were pre-treated with the following surface modification method to form hydrophilic surfaces of channels.
1. Plates were washed with 99.8% methanol (Sigma-Aldrich #322415) in Ultrasonic Cleaner (GemOro Model: 10 QTH) for 15 min.
2. Plates were washed with milli-Q water in the Ultrasonic Cleaner for 5 min.
3. Plates were treated with 100 mL of 96% Sulfuric Acid (Sigma-Aldrich #7664-93-9) and 5 g of NOCHROMIX (Sigma #328693) mixture for 60 min.
4. Step “2” was repeated.
5. Plates were washed with 1 mg/mL of Sodium Hydroxide (Fisher Chemical CAS 1310-73-2) in the Ultrasonic Cleaner for 30 min.
6. Step “2” was repeated.
7. Plates were incubated with 0.2 mg/mL of Polyethyleneimine solution (Sigma-Aldrich #181978-5G) for 30 min.
8. Plates were washed with milli-Q water for 5 min.
9. Plates were incubated with 1% of Glutaraldehyde solution (Fisher Chemical CAS Registry Number1: 111-30-8, CAS Registry Number2: 7732-18-5) for 15 min.
10. Step “8” was repeated.
11. Plates were air dried.

The surface modification method of hydrophobic surface to hydrophilic surface alteration can be substituted with any other well-established techniques such as plasma and radiation methods.

### Reagents Preparation and ELISA Protocol of Optofluidic Well Plate

Human IL-6 DuoSet ELISA Kit (DY206), ELISA Plate-coating buffer (DY006), wash buffer (WA126) and reagent diluent (DY995) were purchased from R&D Systems. The reagents were prepared according to the procedure described in the kits' user manuals. First, the stock wash buffer and reagent diluent were diluted with Milli-Q water to achieve 1× working solutions. Then, the capture antibody working solution of 12 μg/mL was prepared by diluting with PBS (R&D Systems # DY006). The detection antibody working solution of 0.4 μg/mL was prepared by diluting with the 1× Reagent Diluent. The Human IL-6 standard was diluted to a desired concentration by adding the 1× Reagent Diluent as buffer medium and serum. The horseradish peroxidase labeled streptavidin (SAv-HRP) working solutions was prepared just before application by diluting 1 μL of the SAv-HRP stock solution from DY206 kit with 40 μL of the 1× Reagent Diluent. The 1× Reagent Diluent (1% BSA in PBS) was used as a blocking solution.

QuantaRed™ Enhanced Chemifluorescent HRP Substrate Kit (Thermo Scientific #15159) was used at the last step of ELISA protocol to develop fluorescence. The working substrate solution for fluorescence detection was prepared just before application by mixing with 2 μl QuantaRed10-acetyl-3,7-dihydroxyphenoxazine (ADHP) concentrate, 100 μl enhancer solution, and 100 μl stable peroxide solution from the kit at room temperature. In the case of chemiluminescence detection, equal parts of the SuperSignal ELISA Femto Luminol/Enhancer and SuperSignal ELISA Femto Stable Peroxide from SuperSignal™ ELISA Femto Substrate kit were mixed at room temperature just before optical detection step. After preparation of the reagents, following procedure was used for IL-6 detection.

1. Plates were incubated with 10 μL of 12 μg/mL of capture antibody solution for 10 minutes for 0.008 in×0.008 in well plate (20 μL of 4 μg/mL of capture antibody solution for 60 minutes for 0.018 in×0.022 in well plate).
2. Plates were washed with 10 μL of 1× wash buffer for 5 minutes for 0.008 in×0.008 in well plate (20 μL of 1× wash buffer for 1 minute for 0.018 in×0.022 in well plate).
3. Plates were incubated with 10 μL of blocking buffer (1% BSA in PBS) for 10 minutes for 0.008 in×0.008 in well plate (20 μL of blocking buffer for 29 minutes for 0.018 in×0.022 in well plate).
4. Plates were filled with 10 μL of solution (lx Reagent Diluent or serum) containing the standard analyte, IL-6, and incubated for 10 minutes for 0.008 in×0.008 in well plate (20 μL of the standard analyte, IL-6, and incubated for 15 minutes for 0.018 in×0.022 in well plate).
5. Plates were washed with 10 μL of 1× wash buffer for 5 minutes for 0.008 in×0.008 in well plate (20 μL of 1× wash buffer for 1 minute for 0.018 in×0.022 in well plate).
6. Plates were incubated with 10 μL of 0.4 μg/mL of detection antibody solution for 5 minutes for 0.008 in×0.008 in well plate (20 μL of 0.1 μg/mL of detection antibody solution for 15 minutes for 0.018 in×0.022 in well plate).
7. Plates were washed with 10 μL of 1× wash buffer for 5 minutes for 0.008 in×0.008 in well plate (20 μL of 1× wash buffer for 1 minute for 0.018 in×0.022 in well plate).
8. Plates were filled with 10 μL of 1× SAv-HRP solution and incubated for 5 minutes for 0.008 in×0.008 in well plate (10 μL of 1.5×SAv-HRP solution for 5 minutes for 0.018 in×0.022 in well plate).
9. Plates were washed with 10 μL of 1× wash buffer for 5 minutes for 0.008 in×0.008 in well plate (30 μL of 1× wash buffer for 1 minute for 0.018 in×0.022 in well plate).
10. Step 9 was repeated.
11. For fluorescence detection, plates were filled with 3 μL of working substrate solution of QuantaRed™ and the solution was kept still in clear and transparent 0.008 in×0.008 in well plate (8 μL of working substrate solution of QuantaRed™ and the solution was kept still in clear and transparent 0.018 in×0.022 in well plate).
12. Five minutes after Step 10, fluorescence signal was acquired using Standard Multilabel Plate Reader (Perkin Elmer EnSpire® 2300). The reader was set at excitation wavelength of 550 nm with <8 nm bandwidth, emission wavelength of 605 nm with <8 nm bandwidth, and excitation flash intensity of 100. Fluorescence intensity readings were taken from above the well plates. The height of measurement head which consists of excitation and emission channels was adjusted at 11.1 mm to gain maximum sensitivity of the well plate.

In the case of chemiluminescence detection, above steps 11 and 12 are substituted with the following steps 11(A) and 12(A).

11(A). Plates were filled with 8 μL of working substrate solution of SuperSignal™ ELISA Femto and the solution was kept still in black and opaque 0.018 in×0.022 in well plate.
12(B). One minute after Step 10, chemiluminescence signal was acquired using Standard Multilabel Plate Reader (Perkin Elmer EnSpire® 2300). Chemiluminescence intensity readings were taken from below of the well plates.

Note: All measurements reported in this disclosure were performed at room temperature and the solutions were withdrawn using capillary forces.

### Calculation of Results

Data were plotted and calculated mean value, standard deviation, coefficient of variations (CVs), and statistical p-value from triplicate samples (3 wells) of each IL-6 concentration. A four parameter logic (4-PL) was used as a curve fitting. Margin errors at different confidence levels of each concentration were calculated based on number of measurements, degrees of freedom of the data set, values of Student's t, and measured standard deviation. Confidence level (%) was classified by comparing the margin errors with mean differences between blank (0 pg/mL of IL-6) and each concentration.

### Results and Conclusions: Optical Performance

To evaluate optical detection accuracy, fluorescence dye Rhodamine 6G (R6G) solution was used as a model analyte. FIG. 13 exhibits run-to-run variations of 3 wells each from three different well types, (1) conventional 96-well plate, (2) OPTIMISER™ well plate, and (3) optofluidic well plate of embodiments of the present disclosure. (“run-to-run” means that the same well in a plate was read by the plate reader and taken out, and then pushed in and read by the reader again. For each well, the above procedures were repeated 3 times.) Coefficient of variations (CVs) of conventional 96-well plate and optofluidic well plate are similar and less than 5% whereas CVs of OPTIMISER™ well plate are from ˜15 to 35% in 3 μL of R6G (FIG. 13A) and ˜15 to 50% in 10 μL of R6G (FIG. 13B). Indeed, run to run fluoresce reading of a well should not have any variations other than plate reader system variations. Therefore, CVs of 96-well plate are from the reader variations. Consequently, it was concluded that the optofluidic well plate of embodiments of the present disclosure does not introduce any additional variations since CVs of both 96-well and optofluidic plates are similar. On the other hand, it was found that CVs of OPTIMISER™ well plates are 3 to 10 times higher than that of the other two well plates. Thus, the source of the additional CVs is from the plate itself. Furthermore, the usage of 10 μL of R6G introduced more variations than the usage of 3 μL of R6G in the case of OPTIMISER™ well plate, which may be due to more R6G residual in the inlet which locates in the optical excitation and detection path. The design of the plates of embodiment of the present disclosure totally eliminates this problem.

FIG. 14 exhibits well to well variations of 3 runs each from the three different well types. Maximum CVs of conventional 96-well plate and optofluidic well plate are ˜5%. Maximum CVs of OPTIMISER™ well plate are ˜20% in 3 μL of R6G (FIG. 14A) and ˜100% in 10 μL of R6G (FIG. 14B). Again, more variations in the usage of 10 μL of R6G in the case of OPTIMISER™ well plate is due to more R6G residual in the inlet than the usage of 3 μL of R6G. Moreover, asymmetric channel size with spiral structure of OPTIMISER™ well plate needs precise detection position for signal consistency. In contrast, with symmetric channel size with linear U-turn feature of microfluidic channels in the optofluidic well plate of embodiments of the present disclosure accepts large tolerance of optical detection position without signal variation.

FIG. 15 exhibits fluorescence intensity of the same concentration (0.5 μM) of Rhodamine 6G (R6G) with 7 different sizes of channels. The fluorescence intensity increases with bigger channel sizes due to higher optical detection depths. The 0.018 in×0.022 in channel generated over three times higher intensity than that of the 0.008 in×0.008 in channel.

As shown in FIG. 16A, no cross talk (less than 0.25% in all wells) was observed in fluorescence detection using clear and transparent optofluidic well plate. However, significant cross talk (about 10% cross talk in adjacent wells) were observed in chemiluminescence detection using the same transparent well plate (FIG. 16B). In contrast, no cross talk (less than 0.08% in all wells) was found in the case of chemiluminescence detection with a black and opaque optofluidic well plate (FIG. 16C).

### Results and Conclusions: ELISA

Surface modification is an important step before ELISA to alter hydrophobic to hydrophilic surfaces of channels, which allows flow of reagents. Immobilization of capture antibody and blocking of the optofluidic well plate takes 25 minutes for 0.008 in×0.008 in channel (90 minutes for 0.018 in×0.022 in channel) whereas a conventional 96-well plate takes over night. The total assay time of the optofluidic well plate is 45 minutes or less including substrate incubation, whereas a conventional 96-well plate takes about 300 minutes as per user manual from the # DY006 kit. The plate described herein is over 6 times faster than a conventional well plate. Furthermore, the optofluidic well plate needs only 10 μL of analyte sample, which is 10 times less than that used in traditional ELISA. In addition, the optofluidic well plate consumes less reagents (capture antibody, detection antibody, SAv-HRP, and QuantaRed™) than conventional well plate as shown in Table 1.

TABLE 1
Comparison of IL-6 ELISA using Optofluidic
(0.008 in × 0.008 in channel and
0.018 in × 0.022 in channel) and conventional 96-well plates

| | Optofluidic well plate | | | | | |
|---|---|---|---|---|---|---|
| | 0.008 in (W) × | | 0.018 in (W) × | | Conventional | |
| | 0.008 in (D) | | 0.022 in (D) | | 96-well plate | |
| Plate preparation time | 25 | minutes | 90 | minutes | Over night | |
| (coating of capture | | | | | | |
| antibody and blocking) | | | | | | |
| ELISA assay time | 45 | minutes | <45 | minutes | 300 | minutes |
| Analyte sample volume | 10 | μL | 20 | μL | 100 | μL |
| Capture antibody | 0.12 | μg | 0.08 | μg | 0.2 | μg |
| | (un-optimized) | | (un-optimized) | | | |
| Detection antibody | 0.004 | μg | 0.002 | μg | 0.005 | μg |
| | (un-optimized) | | (un-optimized) | | | |
| SAv-HRP | 10 | μL | 10 | μL | 100 | μL |
| Substrate | 3 | μL | 8 | μL | 100 | μL |
| (QuantaRed ™ | | | | | | |
| for fluorescence | | | | | | |
| detection and | | | | | | |
| SuperSignal ™ for | | | | | | |
| chemiluminescence | | | | | | |
| detection) | | | | | | |

The experimental results of IL-6 in log-log scale fit very well with four parameter logic (4-PL) simulated curve. A linear projection was observed between 75 pg/mL and 2400 pg/mL in the log-log scale in both three points plot FIG. 17A and average plot FIG. 17B of 0.008 in×0.008 in channel optofluidic well plates. In FIG. 17C, all coefficient of variations except 0, 37.5, and 600 pg/mL are less than 10%. Only two p values respect to blank (0 pg/mL) of 9.37 pg/mL and 18.75 pg/mL are above cut-off p value (0.05) as shown in FIG. 17D. Thus, those two concentrations are unable to distinguish statistically with the blank. Three p values respect to adjacent lower concentration of 9.37 pg/mL, 18.75 pg/mL, and 75 pg/mL are above cut-off p value. That indicates that these concentrations are unable to distinguish statistically with adjacent lower concentrations (FIG. 13B). Confidence levels of above 95% are found 37.5 pg/mL and above concentrations (Table 2). In summary, a detection limit of 37.5 pg/mL and a detection range between 37.5 pg/mL and 9600 pg/mL were achieved in this IL-6 ELISA with the optofluidic well plate with 0.008 in×0.008 in channel.

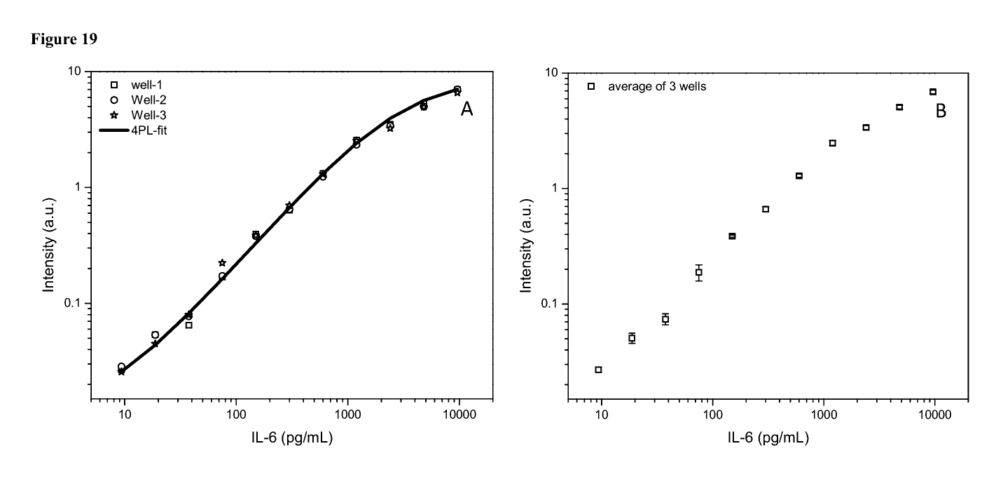

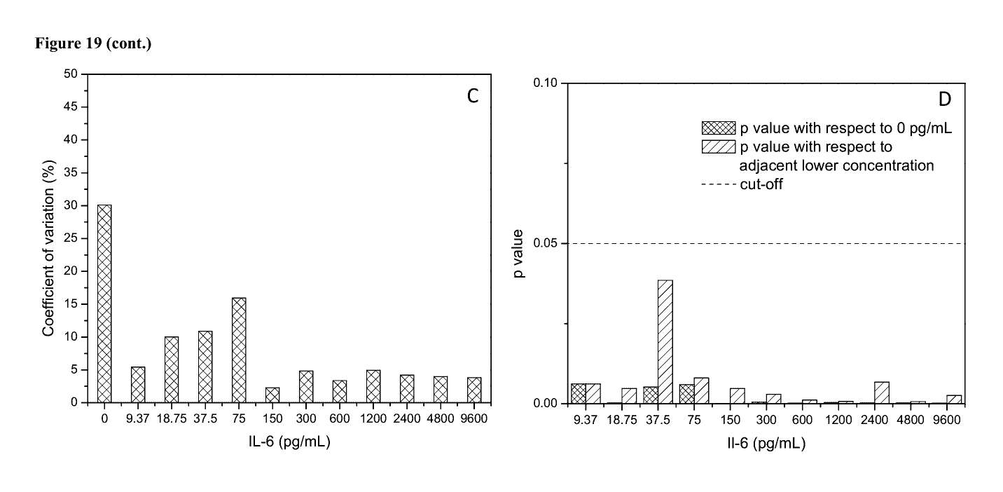

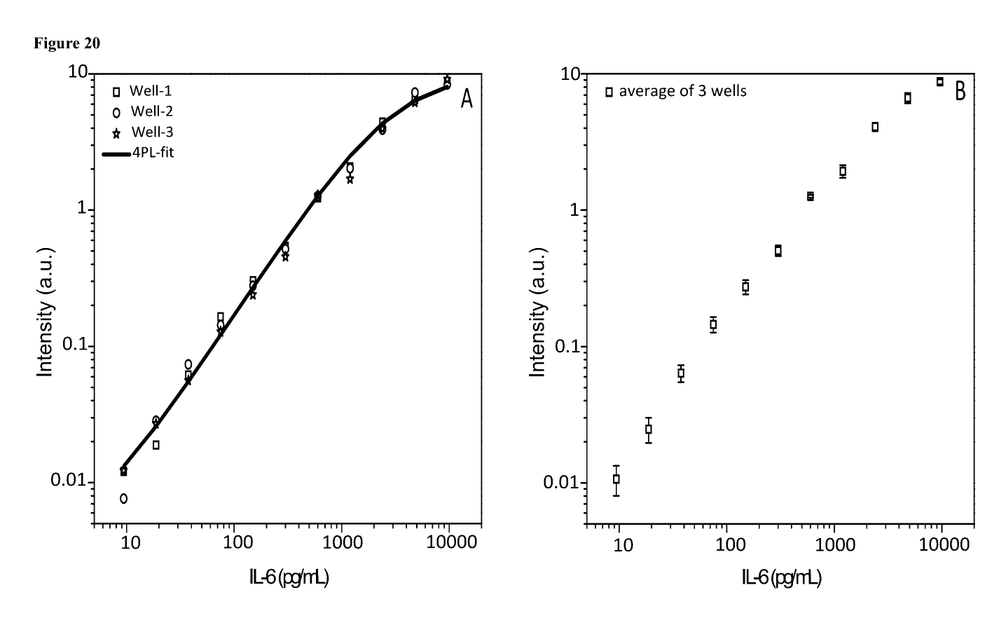

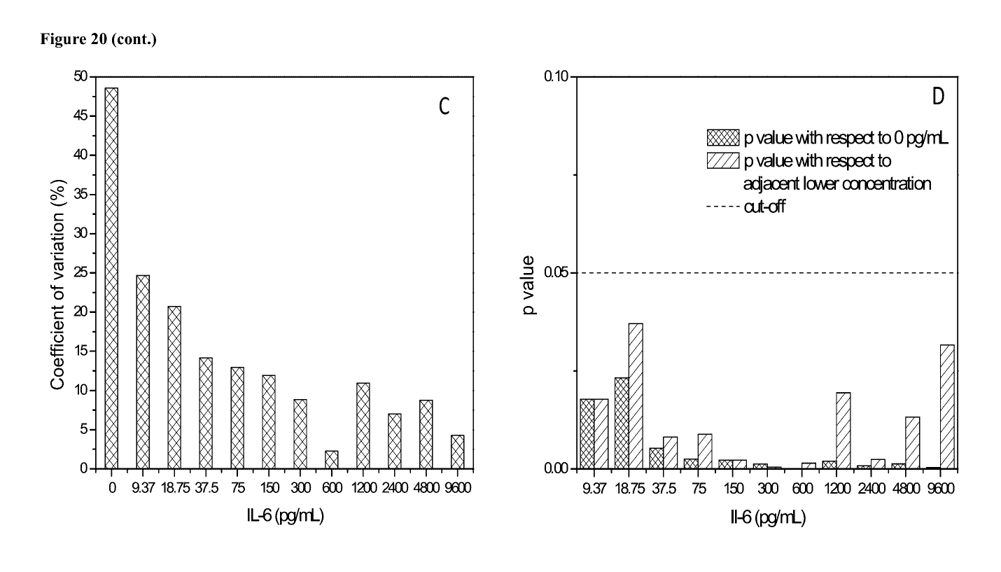

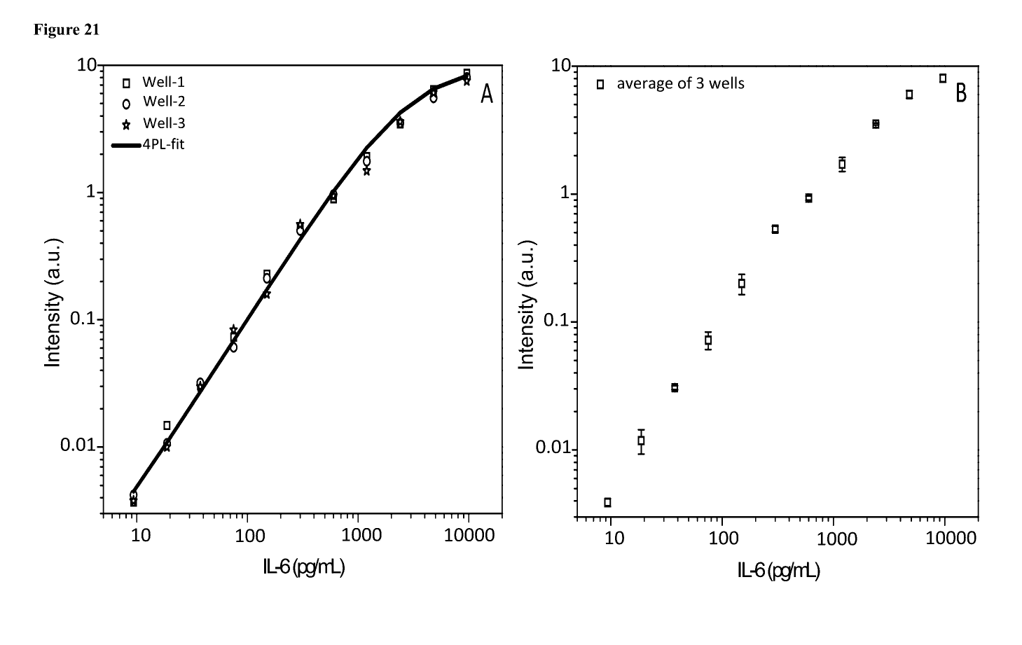

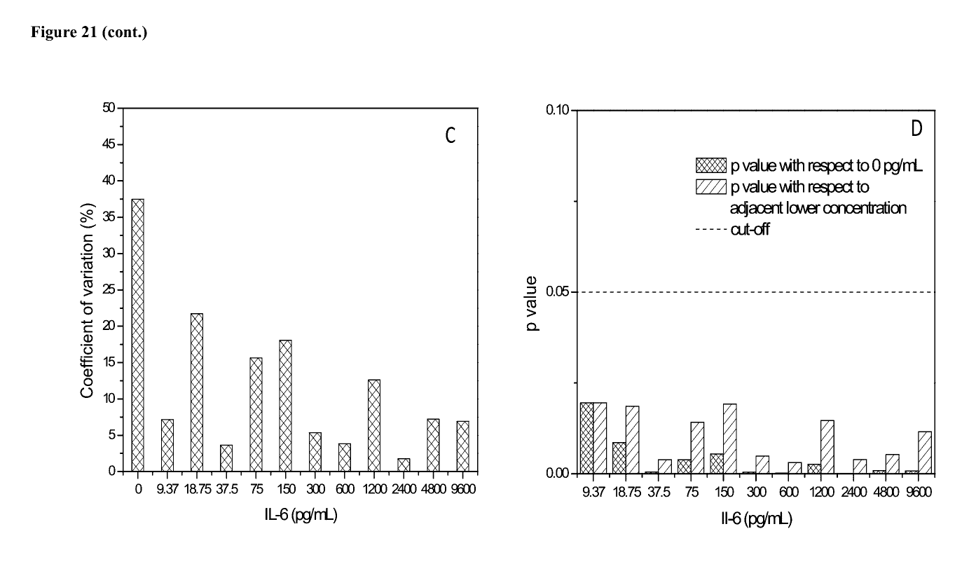

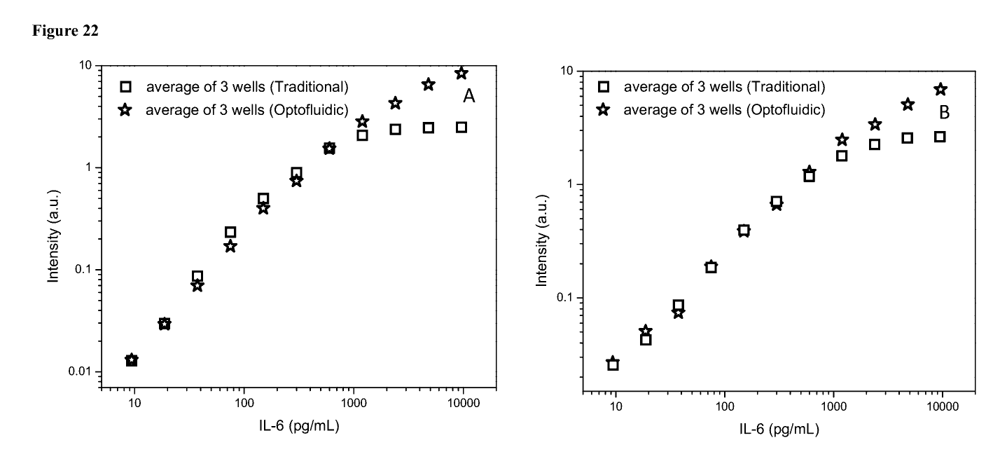

In the case of 0.018 in×0.022 in channel, linear projections were further extended between 9.37 pg/mL and 4800 pg/mL in the log-log scale in both IL-6 with buffer and serum as well as both fluorescence (FIG. 18A, B and FIG. 19A, B) and chemiluminescence detection methods (FIG. 20A, B and FIG. 21A, B). Most of the coefficient of variations were found less than 10% (FIG. 18C, FIG. 19C, FIG. 20C, and FIG. 21C).

The p values of optofluidic well plates with 0.018 in×0.022 in channel improved significantly over 0.008 in×0.008 in channel. All of the p values respect to adjacent lower connections or blank (0 pg/mL) of 0.018 in×0.022 in channel are less than 0.05 (FIG. 18D, FIG. 19D, FIG. 20D, and FIG. 21D). That is statically significance and concentrations between 9.37 pg/mL and 9600 pg/mL of IL-6 are distinguishable from blank (0 pg/mL) as well as adjacent concentrations (eg., 9.37 pg/mL vs. 18.75 pg/mL).

Furthermore, apart from 9.37 pg/mL in buffer of chemiluminescence detection (>90% confidence level), all other concentrations in buffer or serum using fluorescence or chemiluminescence method exhibited confidence levels of above 95% (see Table 2).

Overall, the detection limit is less than 9.37 pg/mL in buffer or serum with both fluorescence and chemiluminescence detection methods using 0.018 in×0.022 in channel. The linear detection range of the 0.018 in×0.022 in channel increased down to 9.37 pg/mL and up to 4800 pg/mL while maintaining the highest detection limit of 9600 pg/mL in line with four parameter logic (4-PL) curve. The statistical p values and confidence levels of 0.018 in×0.022 in channel are much better than 0.008 in×0.008 in channel.

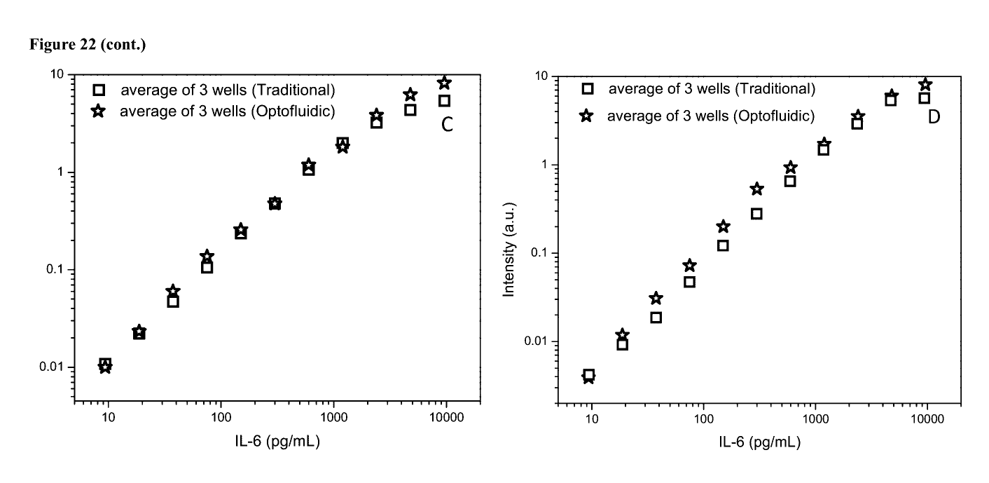

FIG. 22 shows a benchmarking analysis of conventional 96-well plate (about 300 minutes of assay time) with optofluidic well plate (45 minutes or less assay time) using IL-6 in buffer and serum. The highest detection limit of conventional 96-well plate is 1200 pg/mL while optofluidic is 9600 pg/mL in both buffer (FIG. 22A) and serum (FIG. 22B) using fluorescence detection method. In the case of chemiluminescence detection method, both 96-well plate and optofluidic well plate have similar trends as shown FIGS. 22C and D. However, the highest detection limit of 96-well plate is approximately 4800 pg/mL of IL-6 in both buffer (FIG. 22C) and serum (FIG. 22D). On the other hand, optofluidic well plate enabled to detect 9600 pg/mL of IL-6 in both buffer (FIG. 22C) and serum (FIG. 22D).

TABLE 2
Classification of confidence level (%) of each concentration

| | Confidence level (%) | | | | |
|---|---|---|---|---|---|
| | | | | Chemi- | Chemi- |
| | fluorescence | fluorescence | fluorescence | luminescence | luminescence |
| | detection of | detection of | detection of | detection of | detection of |
| | 0.008 in × | 0.018 in × | 0.018 in × | 0.018 in × | 0.018 in × |
| Concentration | 0.008 in | 0.022 in | 0.022 in | 0.022 in | 0.022 in |
| of IL-6 | channel with | channel with | channel with | channel with | channel with |
| (pg/mL) | buffer | buffer | serum | buffer | serum |
| 9.37 | <50 | >98 | >99 | >90 | >99 |
| 18.75 | <50 | >98 | >99 | >95 | >98 |
| 37.5 | >95 | >98 | >99 | >99 | >99.9 |
| 75 | >98 | >99.9 | >99 | >99 | >99 |
| 150 | >99 | >99.9 | >99.9 | >99 | >98 |
| 300 | >99.9 | >99.9 | >99.9 | >99 | >99.9 |
| 600 | >99.9 | >99.9 | >99.9 | >99.9 | >99.9 |
| 1200 | >99.9 | >99.9 | >99.9 | >99 | >99 |
| 2400 | >99.9 | >99.9 | >99.9 | >99 | >99.9 |
| 4800 | >99.9 | >99.9 | >99.9 | >99 | >99 |
| 9600 | >99.9 | >99.9 | >99.9 | >99.9 | >99 |

All publications and patents mentioned in the above specification are herein incorporated by reference in their entirety. Various modifications and variations of the described devices, methods and/or systems will be apparent to those skilled in the art without departing from the scope and spirit of the disclosure. Although the disclosures have been described in connection with specific preferred embodiments, it should be understood that the disclosures as claimed should not be unduly limited to such specific embodiments. Indeed, various modifications of the described modes for carrying out the disclosures which are obvious to those skilled in the relevant fields are intended to be within the scope of the following claims.

We claim:

1. An optofluidic array plate, comprising:
a plurality of wells, wherein each well comprises a liquid inlet; an optically clear fluidic and detection channel comprising a plurality of microposts; and a liquid outlet.

2. The array plate of claim 1, wherein said inlet is offset from said detection channel.

3. The array plate of claim 1, wherein said detection channel comprises a plurality of curves.

4. The array plate of claim 3, wherein said curves are U-shaped.

5. The array plate of claim 1, wherein said detection channel has a symmetrical size.

6. The array plate of claim 1, wherein said inlet is a funnel.

7. The array plate of claim 1, wherein said outlet is a nozzle.

8. The array plate of claim 1, wherein the bottom surface of said array plate comprises a reflective layer.

9. The array plate of claim 1, wherein said array plate comprises 96 wells.

10. The array plate of claim 1, wherein the optically clear channel is transparent in all sides or all directions.

11. The array plate of claim 1, wherein the optically clear channel is transparent in one or two sides or directions, but optically opaque or blocked in the remaining sides or directions.

12. A system, comprising:
a) the array plate of claim 1; and
b) a bottom plate or film configured to attach to the bottom of said array plate, wherein said bottom plate or film comprises a plurality of open-bottom fluidic outlets that correspond to each of said wells.

13. The system of claim 12, wherein said array plate and said bottom plate are hermetically sealed.

14. The system of claim 12, wherein said film is adhesive film with die cut holes.

15. The system of claim 12, wherein said system further comprises a liquid transport pump.

16. The system of claim 12, further comprising a detection component.

17. The system of claim 16, wherein said detection component is a spectrophotometer or plate reader.

18. A method of performing an assay, comprising:
a) contacting a sample suspected of comprising an analyte with the system of claim 12; and
b) performing a detection assay with said system.

19. The method of claim 18, wherein said analyte is a protein or a nucleic acid.

20. The method of claim 18, wherein said assay is an immunoassay, a nucleic acid amplification assay, or a nucleic acid hybridization assay.
